(19) United States
(12) Patent Application Publication
(10) Pub. No.: US 2026/0035657 A1
(43) Pub. Date: Feb. 5, 2026

(54) COMPLEX MICROBIAL INOCULANT FOR EXHAUST GAS TREATMENT BASED ON AEROBIC COMPOSTING, AND PREPARATION METHOD AND APPLICATION THEREOF

(71) Applicant: ZHEJIANG OCEAN UNIVERSITY, Zhoushan (CN)

(72) Inventors: Dongzhi Chen, Zhoushan (CN); Xianwang Kong, Zhoushan (CN); Jianmeng Chen, Zhoushan (CN); Zeyu Yang, Zhoushan (CN); Changwen Chen, Zhoushan (CN)

(21) Appl. No.: 18/861,797

(22) PCT Filed: Dec. 26, 2023
(86) PCT No.: PCT/CN2023/141840

§ 371 Date: Oct. 21, 2025

(30) Foreign Application Priority Data
Mar. 31, 2023 (CN) 2023103336028

(51) Int. Cl. C12N 1/20; B01D 53/84; C12M 1/00; C12M 1/02; C12M 1/06; C12M 1/16; C12N 1/36; C12R 1/38

(52) U.S. Cl. CPC C12N 1/20; B01D 53/84; C12M 21/16; C12M 27/02; C12M 29/04; C12M 41/22; C12N 1/36; B01D 2251/95; C12R 2001/38

(57) ABSTRACT

The present disclosure relates to the technical field of biological treatment of exhaust gases, in particular to preparation of a complex microbial inoculant, an apparatus for preparing the complex microbial inoculant and application thereof. The preparation of the complex microbial inoculant is to select kitchen wastes, wood chips and EM fungus chaff as aerobic composting raw materials, and add contaminant degrading bacteria Pseudomonas mendocin NX-1, Stenotrophomonas sp. HY-2, Rhodococcus sp. YZ-1, Ralstonia sp. XZW-1, and Pseudomonas oleovorans DT4 during the aerobic composting process for joint culture until a compost pile becomes thoroughly decomposed. The complex microbial inoculant provided by the present disclosure can simultaneously degrade n-hexane, pentane, chlorobenzene and tetrahydrofuran, and has the remarkable advantages of simple preparation process, low cost, low carbon, environmental friendliness and the like. The formed microbial inoculant that is rich in efficient degrading bacteria does not need to adjust pH, has the advantages of short starting cycle, and stable and efficient exhaust gas purification, has stronger environmental adaptability, and lays a foundation for engineering application of purifying waste water and exhaust gases containing n-hexane, pentane, chlorobenzene and tetrahydrofuran by a biological method.

### TECHNICAL FIELD

The present disclosure relates to the technical field of biological treatment of exhaust gases, in particular to a complex microbial inoculant for exhaust gas treatment based on aerobic composting, and a preparation method and application thereof.

### BACKGROUND ART

Urban and industrial developments present numerous environmental problems, where volatile organic compounds (VOCs) emitted during industrial production processes are an important class of atmospheric contaminants. Currently, ozone (O₃) and fine particulate matter (PM_{2.5}) are main factors affecting the air quality of China. Studies have shown that VOCs are primary control factors for the generation of O₃ and PM_{2.5} in key regions such as the Beijing-Tianjin-Hebei region and the Yangtze River Delta. Therefore, controlling VOC emissions is crucial for the continued success in the battle to protect the blue sky.

The principle of biological treatment processes of VOC exhaust gases is to convert gaseous contaminants into innocuous substances, such as CO₂, H₂O, lowly toxic intermediate products, and cytoplasm under the metabolism of microorganisms. Compared with physicochemical technologies such as adsorption and combustion, the biological treatment processes have the advantages of mild reaction conditions, small secondary pollution, low carbon emissions and the like.

Microorganisms with VOCs degrading capability are the core of the biological treatment processes. Complex microbial inoculants are a mixed bacterium culture system constructed by taking high-activity degradation strains as materials, and can efficiently degrade contaminants with complex components under the independent action or the inter-species synergistic action of the microorganisms.

At present, the problems of the prepared complex microbial inoculants mainly include complex preparation process, low degradation activity of microbial inoculants, finite viable count of microbial inoculants per unit volume and the like. Thus, there is an urgent need to develop a new complex microbial inoculant to meet higher requirements in current production practice.

### SUMMARY

The present disclosure provides a complex microbial inoculant for exhaust gas treatment based on aerobic composting, and a preparation method and application thereof in order to overcome the defects in the prior art. According to the present disclosure, aerobic composting and microbial inoculant preparation are combined, VOCs degrading bacteria are gradually cultured using natural carriers and various nutrient elements provided by a compost pile, such that the synchronous implementation of the aerobic composting and the microbial inoculant preparation is realized, thus forming a technology of the solid complex microbial inoculant with the VOCs degrading capability. The preparation process is simple, and the preparation method has important practical significance for the efficient treatment of gaseous contaminants.

In order to fulfill the above objectives, the present disclosure provides the following technical solution:

A preparation method of a complex microbial inoculant for exhaust gas treatment based on aerobic composting includes:
selecting Pseudomonas mendocina NX-1, Stenotrophomonas sp. HY-2, Rhodococcus sp. YZ-1, Ralstonia sp. XZW-1, and Pseudomonas oleovorans DT4, wherein the Pseudomonas mendocina NX-1 and the Stenotrophomonas sp. HY-2 are screened through n-hexane serving as a carbon source, the Rhodococcus sp. YZ-1 is screened through pentane serving as a carbon source, the Ralstonia sp. XZW-1 is screened through chlorobenzene serving as a carbon source, and the Pseudomonas oleovorans DT4 is screened through tetrahydrofuran serving as a carbon source; inoculating the above strains into an inorganic salt solid medium, then respectively inoculating the five strains into an LB broth for culture to obtain an inoculum solution, inoculating the inoculum solution onto kitchen wastes serving as a culture medium, introducing organic exhaust gases through aerobic composting, and carrying out a domestication and culture process to obtain the complex microbial inoculant; wherein
the Pseudomonas mendocin NX-1, deposited in the China Center for Type Culture Collection, located at the University of Wuhan, 430072, Wuhan, China, with an accession number of the deposit: CCTCC No. M2015114, is a known strain that has been disclosed in CN105087440A;
The Stenotrophomonas sp. HY-2, deposited in the China Center for Type Culture Collection, located at the University of Wuhan, 430072, Wuhan, China, with an accession number of the deposit: CCTCC No. M2018714, is a known strain that has been disclosed in 109536413A;
the Rhodococcus sp. YZ-1, deposited in the China Center for Type Culture Collection, located at the University of Wuhan, 430072, Wuhan, China, with an accession number of the deposit: CCTCC No. M20221106, is a known strain that has been disclosed in CN116004459A;
the Ralstonia sp. XZW-1, deposited in the China Center for Type Culture Collection, located at the University of Wuhan, 430072, Wuhan, China, with an accession number of the deposit: CCTCC No. M2022557, is a known strain that has been disclosed in CN115838651A; and
the Pseudomonas mendocin DT4, deposited in the China Center for Type Culture Collection, located at the University of Wuhan, 430072, Wuhan, China, with an accession number of the deposit: CCTCC No. M209151, is a known strain that has been disclosed in CN101845408A.

During the early experimental process, the inventor of the present application screened out strains having excellent degradation effects on n-hexane, pentane, chlorobenzene and tetrahydrofuran, namely, the Pseudomonas mendocin NX-1 and the Stenotrophomonas sp. HY-2 having the excellent degradation effect on the n-hexane, the Rhodococcus sp. YZ-1 having the excellent degradation effect on the pentane, the Ralstonia sp. XZW-1 having the excellent degradation effect on the chlorobenzene and the Pseudomonas oleovorans DT4 having the excellent degradation effect on the tetrahydrofuran. The strains are characterized by growing respectively by taking the n-hexane/the pentane/the chlorobenzene/the tetrahydrofuran as the unique carbon source and energy, and meanwhile, efficiently degrading the substrates. While the contaminants of the n-hexane, the pentane, the chlorobenzene, and the tetrahydrofuran are several common VOCs, which primarily derive from tail gases emitted by petrochemical industry, coating industry, pharmaceutical factories, etc. and pose great harm to human bodies and environments.

For the convenience of an experiment, organic exhaust gases are prepared by mixing the n-hexane, the pentane, the chlorobenzene and the tetrahydrofuran.

In the experiment, the inoculum solution is prepared from the five strains, and with aerobic composting raw materials (kitchen wastes) as a “culture medium”, the inoculum solution is then inoculated onto the “culture medium”, the organic exhaust gases are introduced to domesticate the degrading bacteria such as the Pseudomonas mendocin NX-1, the Stenotrophomonas sp. HY-2, the Rhodococcus sp. YZ-1, the Ralstonia sp. XZW-1, and the Pseudomonas oleovorans DT4, and finally, the solid complex microbial inoculant is obtained through culture. The degrading bacteria may grow by taking the kitchen wastes as the carbon source, and may also grow by taking the organic exhaust gases as the carbon source, without competition from other microorganisms when the organic exhaust gases serve as the carbon source. The organic exhaust gases play a role in domesticating microflorae in a composting system. The degrading bacteria may prepare the solid complex microbial inoculant and simultaneously carry out composting treatment on the kitchen wastes, such that aerobic composting and complex microbial inoculant preparation may be synchronously realized.

The prepared complex microbial inoculant is obtained by culturing bacterial solutions of the Pseudomonas mendocin NX-1, the Stenotrophomonas sp. HY-2, the Rhodococcus sp. YZ-1, the Ralstonia sp. XZW-1, and the Pseudomonas oleovorans DT4 serving as the inoculum solution, and theoretically has high degradation capability on the n-hexane, the pentane, the chlorobenzene and the tetrahydrofuran which are common in VOCs.

As proved by the experiment, the complex microbial inoculant has excellent degradation performance on 100 mg/L n-hexane, pentane, chlorobenzene and tetrahydrofuran: the chlorobenzene and the tetrahydrofuran may be completely degraded within 44 hours, and the n-hexane and the pentane may be completely degraded within 48 hours. Additionally, the continuous contaminant degradation experiment on the complex microbial inoculant shows that the complex microbial inoculant exhibits the excellent continuous degradation performance on various contaminants. Specifically, after degrading 100 mg/L contaminants completely for the first time, the microbial inoculant may completely degrade 100 mg/L chlorobenzene within 6-8 hours in subsequent cycles. After repeating this process 10 times, the concentration of the contaminants is elevated to 200 mg/L, and the contaminants may be completely degraded within 8-10 hours. Meanwhile, the complex microbial inoculant has excellent tolerance to the concentration of the contaminants, and specifically, 100 mg/L and 200 mg/L contaminants may be completely degraded within 50 hours, while the contaminants at the concentrations of 300 mg/L and 400 mg/L achieve a degradation rate of 90% or above within 65 hours.

In conclusion, it could be proved that the prepared complex microbial inoculant may be put into degradation of VOCs, and has important practical significance for efficient treatment of gaseous contaminants. In the solution, not only the microbial inoculant for mineralizing and treating the VOCs may be obtained through preparation, and meanwhile, the minimized, harmless and recycling treatment of the kitchen wastes may also be realized.

Preferably, the inoculum solution is prepared by respectively inoculating five strains for culture: the Pseudomonas mendocin NX-1, the Stenotrophomonas sp. HY-2, the Rhodococcus sp. YZ-1, the Ralstonia sp. XZW-1 and the Pseudomonas oleovorans DT 4,followed by centrifuging, washing, diluting five bacterial suspensions in a logarithm growth period, and mixing five yielded bacterial solutions.

The growth rate of the strains in the logarithm growth period reaches a constant maximum specific growth rate, and at this moment, the strains have high bacterial load and good bacterial activity. The strains are organic exhaust gas (n-hexane, pentane, tetrahydrofuran and chlorobenzene) degrading bacteria, and do not affect the aerobic composting. If the culture is insufficient, the bacterial load added into the whole composting system is low, resulting in the poor degradation effect of the composting microbial inoculant on the contaminants (n-hexane, pentane, tetrahydrofuran and chlorobenzene).

Preferably, the inoculum solution is added to the kitchen wastes in batches.

During the aerobic composting process, a temperature of a compost pile will raise to 50-60° C., and a pH value will be reduced to about 3-4, which will be unfavorable for the inoculation and growth of the strains. How to overcome unfavorable conditions such as excessively high temperatures and overly acidic pH levels is a key problem in the preparation of the complex microbial inoculant for exhaust gas treatment based on aerobic composting. Selecting appropriate inoculation time may aid in the growth of the strains. Given that the inoculation of the inoculum solution is not advisable in the high temperature period, the inventor of the present application chose to perform inoculation outside of the high temperature period.

Based on the 35-day duration of the aerobic composting, the inoculum solution of the complex microbial inoculant is inoculated on Day 1, Day 14, Day 21, Day 27, and Day 34. The first inoculation is performed at the commencement of composting; the second inoculation is performed at the end of the high temperature period of composting (14 days later): in the high temperature period, the excessively high temperatures in the composting system may cause partial inactivation of the inoculated bacteria, and thus an additional inoculation is performed following the conclusion of the high temperature period. Subsequent inoculations are performed at periodic intervals to maximize the number of the degrading bacteria within the composting system, thereby ensuring that the prepared composting microbial inoculant is more effective.

It is noted that the specific duration of the aerobic composting is a cycle required for the fermentation of the compost pile to reach a thoroughly decomposed state, which varies over diversified factors, and the inventor of the present application has enumerated only one specific cycle for illustration.

Preferably, during the aerobic composting process, an amount of the inoculum solution of the complex microbial inoculant that is inoculated every time is 2% (V/W) of a dry weight of the kitchen wastes.

The amount of the inoculum solution at each inoculation is set so as to ensure that the amount of the added bacterial solutions has no more than 1% of influence on the water content.

Preferably, the organic exhaust gases are introduced at the end of the high temperature period of the aerobic composting.

In the high temperature period of the aerobic composting, the activity of the microorganisms related to the composting is intense, and introducing the organic exhaust gases may influence the balance of the system (the inactivation of part of the microorganisms related to the composting), such that internal microflorae are destroyed, resulting in a failure in the aerobic composting. Therefore, the organic exhaust gases are introduced at the end of the high temperature period of the aerobic composting.

Preferably, during the aerobic composting process, stirring is not performed for the first 7 days, and is then performed every 2 days following 7 days.

A stirring frequency is set to no stirring for the first 7 days, followed by stirring every 2 days thereafter. An aeration rate is maintained at 0.5 L/(kg min), where kg refers to the dry weight of the compost pile. This procedure aims to meet the requirements for pathogen elimination in the high temperature period of composting, and to prevent the compaction of the compost pile or the generation of localized anaerobic conditions.

Preferably, besides the kitchen wastes, a conditioner and a kitchen fermentation microbial inoculant are further added during the aerobic composting process.

The conditioner may be any one of wood chips, straw and rice hulls, and the primary objective is to adjust the water content, carbon-nitrogen (C/N) ratio, porosity and the like of the system. Generally, for every 25 g of organic carbon consumed by the microorganisms, 1 g of nitrogen needs to be absorbed. If the C/N ratio is too high, the microbial reproduction rate decreases, and the decomposition of organic matter slows down. If the C/N ratio is too low, excess nitrogen is released in the form of ammonia, resulting in an unpleasant odor.

The kitchen fermentation microbial inoculant is EM fungus chaff. The EM fungus chaff may provide a plurality of strains (such as Bacillus, Lactobacillus and Saccharomyces) which may strongly decompose the organic matter, such that the aerobic composting process is accelerated.

According to the present disclosure, the wood chips, the EM fungus chaff and the kitchen wastes are mixed at a dry weight ratio of 5:1:8 to prepare the aerobic composting raw materials.

A complex microbial inoculant is prepared using the above method. The complex microbial inoculant includes Pseudomonas mendocin NX-1, Stenotrophomonas sp. HY-2, Rhodococcus sp. YZ-1, Ralstonia sp. XZW-1, and Pseudomonas oleovorans DT4.

The preparation of the complex microbial inoculant and aerobic composting synchronously occur. The inventor of the present application carried out the following steps of preparing an inoculum solution from five strains according to the above method, inoculating the inoculum solution onto a “culture medium” by taking aerobic composting raw materials (kitchen wastes) as the “culture medium”, introducing organic exhaust gases to domesticate degrading bacteria such as the Pseudomonas mendocin NX-1, the Stenotrophomonas sp. HY-2, the Rhodococcus sp. YZ-1, the Ralstonia sp. XZW-1, and the Pseudomonas oleovorans DT4, and finally, obtaining the solid complex microbial inoculant through culture. Therefore, the complex microbial inoculant derives from five single bacteria: the Pseudomonas mendocin NX-1, the Stenotrophomonas sp. HY-2, the Rhodococcus sp. YZ-1, the Ralstonia sp. XZW-1, and the Pseudomonas oleovorans DT4.

An apparatus for preparing a complex microbial inoculant includes a gas distribution system for preparing exhaust gases and a solid fermentation tank connected with the gas distribution system for treating the exhaust gases, wherein the gas distribution system includes an air compressor, and an exhaust gas supply bottle and a gas mixing bottle, the exhaust gas supply bottle and the gas mixing bottle are sequentially connected with the air compressor, one side of the air compressor is connected with the exhaust gas supply bottle, the other side of the air compressor is connected with the gas mixing bottle, a thermostat is arranged on an outer wall of the exhaust gas supply bottle, a temperature probe is arranged in the exhaust gas supply bottle, the solid fermentation tank includes a motor, a stirring apparatus is arranged in the solid fermentation tank and connected with the motor, a temperature control jacket is arranged on an outer wall of the solid fermentation tank, and a gas distribution plate is arranged on an inner wall of the solid fermentation tank.

One path of air in the air compressor is introduced into the exhaust gas supply bottle, and the other path of air is mixed with gases from the exhaust gas supply bottle in the gas mixing bottle and then enters the solid fermentation tank. Wherein, organic exhaust gases may be diluted by introducing the air. The thermostat for controlling a temperature of the exhaust gas supply bottle is arranged outside the exhaust gas supply bottle, and the temperature probe for detecting temperature changes in the exhaust gas supply bottle is arranged inside the exhaust gas supply bottle.

The solid fermentation tank serves as an integrated device for aerobic composting of kitchen wastes and a compost pile for substrate domestication. The temperature control jacket designed to maintain a stable and constant temperature is arranged on the outer wall of the solid fermentation tank, while the gas distribution plate designed to achieve the uniform distribution of gases within a tank body is arranged on the inner wall of the solid fermentation tank.

Preferably, a microbial filter is arranged between the gas distribution system and the solid fermentation tank, a mass flowmeter is arranged between the air compressor and the exhaust gas supply bottle, and a rotameter is arranged between the gas mixing bottle and the microbial filter.

The microbial filter is configured to filter microorganisms to avoid microbial contamination in environments.

The mass flowmeter is additionally arranged between the air compressor and the exhaust gas supply bottle, which allows for the regulation and control of air intake, thus controlling the concentration of organic exhaust gases. On one hand, this arrangement prevents excessively high concentrations of the organic exhaust gases from affecting the microbial activity and potentially harming the microorganisms. On the other hand, it avoids excessively high velocity of airflow, which could lead to accelerated loss of temperature and humidity in the compost pile, resulting in reducing the recycling of the compost pile.

The rotameter is additionally arranged between the gas mixing bottle and the microbial filter to control the flow of mixed gases entering the solid fermentation tank, thereby uniformly providing mixed exhaust gases for the tank body. When composting is completed, and the organic exhaust gases are not required to be introduced, only the mass flowmeter is required to be turned off.

Preferably, the stirring apparatus includes a stirring rod and a plurality of stirring paddles arranged on the stirring rod.

Preferably, a gas collection port is formed between the microbial filter and the solid fermentation tank.

Application of the prepared complex microbial inoculant and the apparatus for preparing the complex microbial inoculant in degradation of kitchen wastes and exhaust gas treatment.

Wherein post-treatment methods and deposition temperatures of the prepared complex microbial inoculant will pose an impact on a degradation effect of contaminants. More specifically, the prepared complex microbial inoculant may be used for subsequent contaminant degradation application through methods such as freeze-drying, vacuum-drying, or without any dehydration treatment. In practical applications, the degradation effect is as follows: no dehydration treatment is better than freeze-drying and is approximately equal to vacuum-drying. The impact of the deposition temperature on contaminant degradation depends on categories of the contaminants, with different contaminants being affected to varying degrees. Therefore, the present disclosure has the following beneficial effects:
(1) According to the present disclosure, the kitchen wastes serve as the culture medium, the organic exhaust gases are introduced, and the complex microbial inoculant capable of treating the VOCs is obtained through culture, such that a new idea is provided for the harmless treatment of the gaseous contaminants;
(2) according to the present disclosure, the aerobic composting of the kitchen wastes and the preparation of the complex microbial inoculant capable of treating the VOCs may be synchronously realized;
(3) according to the complex microbial inoculant prepared by the present disclosure, the kitchen wastes are directly used as a microbial inoculant carrier, and other additional chemicals are not required to be added during the preparation process, such that the preparation method is simple in operation, low in cost, low in carbon and environmentally friendly; and
(4) the complex microbial inoculant prepared by the present disclosure may be deposited for a long time at a normal temperature, and has the advantages of no need for pH adjustment, short starting cycle, and stable and efficient exhaust gas purification during the actual degradation application process.

### BRIEF DESCRIPTION OF THE DRAWINGS

FIG. 1 is a schematic structural diagram illustrating an apparatus for preparing a complex microbial inoculant;

FIG. 2 is a graph illustrating results of test experiments in a contaminant degradation effect for a solid complex microbial inoculant;

FIG. 3 is a graph illustrating results of test experiments in a continuous contaminant degradation effect for a solid complex microbial inoculant;

FIG. 4 is a graph illustrating results of test experiments in a contaminant concentration tolerance for a solid complex microbial inoculant; and

FIG. 5 is a graph illustrating results of test experiments in an impact of different treatment methods on a degradation effect of a solid complex microbial inoculant.

Numbers in the figures are as follows: gas distribution system 100; air compressor 110; exhaust gas supply bottle 120; thermostat 121; temperature probe 122; gas mixing bottle 130; mass flowmeter 140; rotameter 150; solid fermentation tank 200; stirring apparatus 210; stirring paddle 211; stirring rod 212; motor 220; temperature control jacket 230; gas distribution plate 240; feed port 250; gas outlet 260; sampling port 270; base 280; supporting post 281; discharge valve 290; microbial filter 300; and gas collection port 400.

### DETAILED DESCRIPTION OF THE EMBODIMENTS

The present disclosure will be further described below with reference to the examples. Those ordinarily skilled in the art can implement the present disclosure based on these descriptions. Furthermore, the examples of the present disclosure involved in the following description are merely a part of examples of the present disclosure, and not all of the examples. Therefore, all other examples obtained by those ordinarily skilled in the art based on the examples in the present disclosure without involving inventive efforts should fall within the protection scope of the present disclosure.

### Example 1 [Apparatus for Preparing Complex Microbial Inoculant]

As shown in FIG. 1, a gas distribution system 100 consists of an air compressor 110, an exhaust gas supply bottle 120, a gas mixing bottle 130, a mass flowmeter 140 and a rotameter 150, wherein one path of air in the air compressor 110 is controlled by the mass flowmeter 140 to be introduced into the exhaust gas supply bottle 120, the concentration of exhaust gases is adjusted by the mass flowmeter 140, and the other path of air is mixed with gases from the exhaust gas supply bottle 120 in the gas mixing bottle 130, and then the flow of the mixed gases entering a solid fermentation tank 200 is controlled by the rotameter 150.

The exhaust gas supply bottle 120 maintains the stability of the concentration of the exhaust gases through a constant temperature blow-off method; a thermostat 121 for controlling the temperature of the exhaust gas supply bottle 120 is arranged outside the exhaust gas supply bottle 120, a temperature probe 122 for detecting temperature changes in the exhaust gas supply bottle 120 is arranged in the exhaust gas supply bottle 120, and the gas mixing bottle 130 for diluting and uniformly mixing the high-concentration organic exhaust gases with the air is sequentially connected with the rotameter 150, a microbial filter 300 and the solid fermentation tank 200. The microbial filter 300 may be used to filter microorganisms to avoid microbial contamination in environments, and a gas collection port 400 is formed between the microbial filter 300 and the solid fermentation tank 200.

The solid fermentation tank 200 serves as an integrated device for aerobic composting of organic solid wastes and a compost pile for substrate domestication, a motor 220, gas outlets 260 and a feed port 250 are arranged on a top of the integrated device, and stirring paddles 211 connected with the motor 220, a gas distribution plate 240 positioned on an inner wall of a conical portion of the solid fermentation tank 200 and a discharge valve 290 positioned at a bottom end of the solid fermentation tank 200 are arranged inside the integrated device. The gas distribution plate 240 may provide assistance in achieving the uniform distribution of the compost pile and gaseous contaminants within a tank body. A temperature control jacket 230 with a temperature sensor is arranged on an outer wall of the solid fermentation tank 200, and a sampling port 270 is formed in the outer wall of the solid fermentation tank, wherein the temperature control jacket 230 may be used for controlling a condensate water valve and a heater switch, so that the jacket may be controlled at a constant temperature. Aerobic composting raw materials are uniformly mixed and then enter the solid fermentation tank 200 from the feed port 250. The solid fermentation tank 200 is vertically fixed to a base 280 through supporting posts 281, the motor 220 is used for longitudinally stirring, and the motor 220 is connected with a plurality of stirring paddles 211 which are transversely arranged through a stirring rod 212.

The gas distribution system 100 continuously provides the organic exhaust gases and oxygen for the compost pile to realize the regulation and control of the concentration and ventilation of the organic exhaust gases. On one hand, the gas distribution system prevents excessively high concentrations of the organic exhaust gases from affecting the microbial activity and potentially harming microorganisms. On the other hand, it avoids excessively high velocity of airflow, which could lead to accelerated loss of temperature and humidity in the compost pile, resulting in reducing the recycling of the compost pile.

### Example 2 [Preparation of Complex Microbial Inoculant]

### 1. Preparation of Culture Medium

### (1) Preparation of Inorganic Salt Solid Medium

The inorganic salt solid culture medium is prepared according to the following formula: 0.023 g/L CaCl₂, 0.2 g/L MgSO₄, 2.5 g/L (NH₄)₂SO₄, 1.0 g/L KH₂PO₄, 4.5 g/L Na₂HPO₄, and 1 mL/L trace elements. The composition of the trace elements: 1 g/L FeSO₄·7H₂O, 0.02 g/L CuSO₄·5H₂O, 0.014 g/L H₃BO₃, 0.10 g/L MnSO₄·4H₂O, 0.10 g/L ZnSO₄·7H₂O, 0.02 g/L Na₂MoO₄·2H₂O, and 0.02 g/L CoCl₂·6H₂O.

(2) Preparation of LB liquid medium

The LB liquid medium is prepared according to the following formula: 5 g/L yeast powder, 10 g/L peptone, and 10 g/L NaCl.

### 2. Preparation of Complex Microbial Inoculant

### (1) Preparation of Inoculum Solution:

Pseudomonas mendocina NX-1, Stenotrophomonas sp. HY-2, Rhodococcus sp. YZ-1, Ralstonia sp. XZW-1, and Pseudomonas oleovorans DT4 were selected, wherein the Pseudomonas mendocina NX-1 and the Stenotrophomonas sp. HY-2 were screened through n-hexane serving as a carbon source, the Rhodococcus sp. YZ-1 was screened through pentane serving as a carbon source, the Ralstonia sp. XZW-1 was screened through chlorobenzene serving as a carbon source, and the Pseudomonas oleovorans DT4 was screened through tetrahydrofuran serving as a carbon source; the above strains were inoculated into an inorganic salt solid medium, then the five strains were respectively inoculated into an LB broth, and was cultured on a shaker through shaking at a temperature of 30° C. for 24 to 48 hours to obtain five bacterial suspensions in a logarithm growth period. The obtained five bacterial suspensions were centrifuged at 8000 rpm/min for 15 minutes, bacterial cells were collected, and washed with deionized water two to three times, and the bacterial solutions were diluted until the bacterial solution concentration was not less than 2.0×10⁹ CFU/mL. The five bacterial solutions that were obtained after centrifugation, washing and dilution were mixed in an equal volume to obtain the inoculum solution of the complex microbial inoculant.

(2) Formation of Complex Microbial inoculant:

In a composting apparatus, kitchen wastes were taken as raw materials, and wood chips and EM fungus chaff (the viable count was approximately 20×10⁹ CFU/g) were added for aerobic composting. Wherein wet weights of the kitchen wastes, the wood chips and the EM fungus chaff were respectively 20 kg, 3.5 kg and 1 kg, a final carbon-nitrogen ratio was 30, and a final water content was 66%. Particle sizes of the kitchen wastes are 1-2 cm, particle sizes of the wood chips are 0.25 cm, and a particle size of the fungus chaff is 0.85 cm.

The aerobic composting was carried out for 35 days, and the prepared inoculum solution was inoculated on Day 1, Day 14, Day 21, Day 27 and Day 34 of the aerobic composting in an inoculation amount that was 2% (V/W) of a dry weight of initial materials (i.e., kitchen wastes). Wherein during the aerobic composting process, stirring was not performed for the first 7 days, and was then performed every 2 days thereafter.

At the end of a high temperature period of the aerobic composting (i.e. Day 14), organic exhaust gases and air were uniformly mixed and then introduced into a composting apparatus, and degrading bacteria (i.e., degrading bacteria generated by seed inoculation in the inoculum solution) in a compost pile were domesticated through the organic exhaust gases until the compost pile became completely decomposed, such that a solid complex microbial inoculant was generated.

### Performance Test

### 1. Determination of the Number of Contaminant Degrading Bacteria in Complex Microbial Inoculant

10 g of the complex microbial inoculant was mixed with 100 g of deionized water, a mixture was put into a shaker at a temperature of 30° C. and a revolving speed of 160 r/min for shaking for 20 minutes, taken out, and allowed to stand for 10 minutes, a supernatant was collected, and counting was performed on a plate through a method of “dipping for liquid phase and volatilizing for gas phase” using the inorganic salt solid medium, wherein results are shown in Table 1.

TABLE 1
Summary table of the number of contaminant degrading
bacteria in complex microbial inoculant

| | | | Tetrahy- | Chloro- |
|---|---|---|---|---|
| | n-hexane | Pentane | drofuran | benzene |
| Number of colonies | 80 | 4 | 1.2 | 20 |
| (×10⁸ CFU/g) | | | | |

2. Contaminant degradation test of complex microbial inoculant

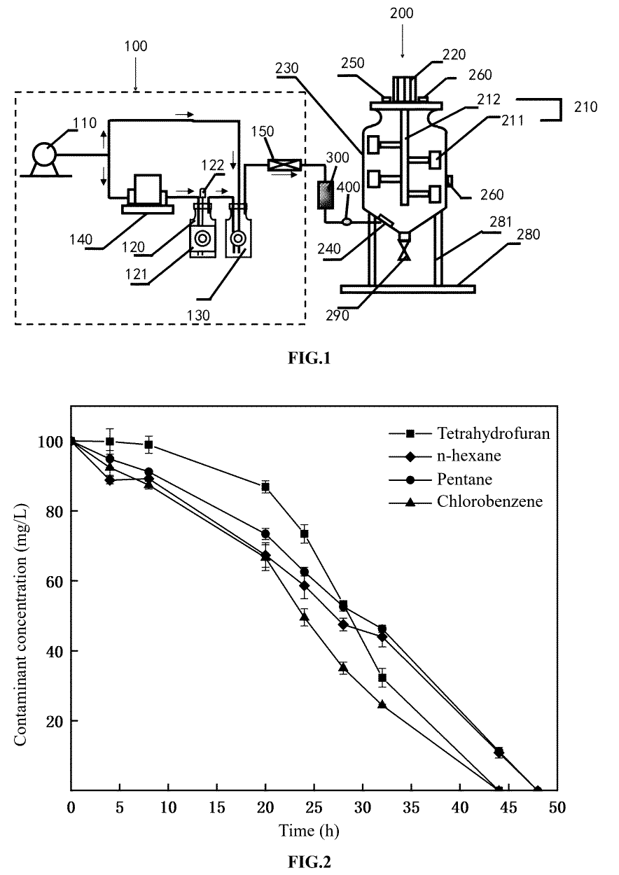

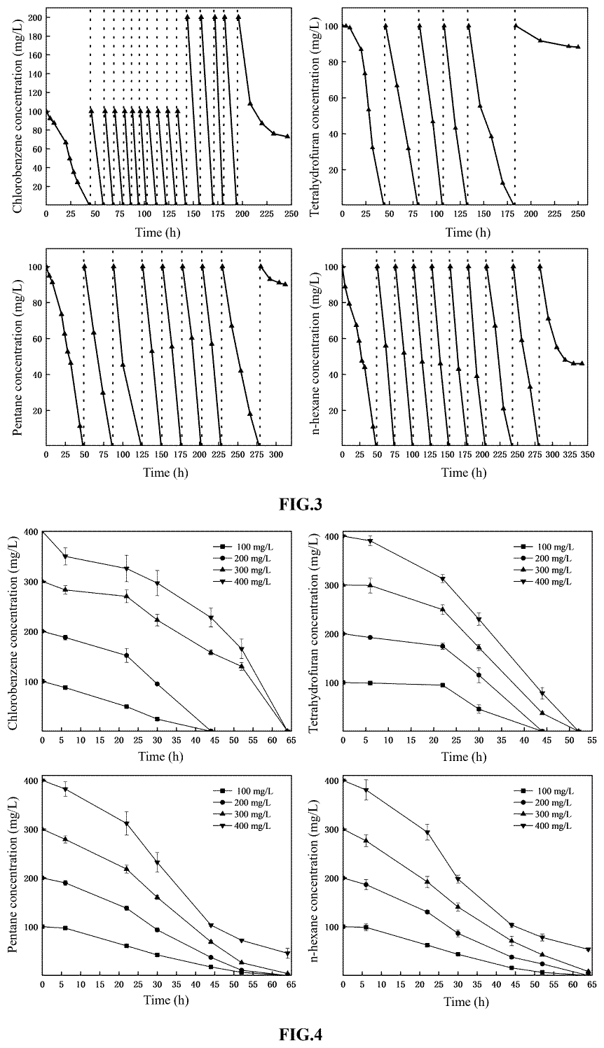

A plurality of parts of 1 g (dry weight basis) of solid microbial inoculant were taken and respectively inoculated into fresh 50 mL inorganic salt media respectively containing 100 mg/L n-hexane, 100 mg/L pentane, 100 mg/L chlorobenzene and 100 mg/L tetrahydrofuran, and the culture medium was placed into a shaker at a temperature of 30° C. and a revolving speed of 160 r/min for culture, and the degradation rate of the n-hexane, the pentane, the chlorobenzene and the tetrahydrofuran was determined, wherein results are shown in FIG. 2.

It could be seen that the complex microbial inoculant had excellent degradation performance on 100 mg/L contaminants, the chlorobenzene and the tetrahydrofuran may be completely degraded within 44 hours, and the n-hexane and the pentane may be completely degraded within 48 hours.

### 3. Continuous Contaminant Degradation Test of Complex Microbial Inoculant

A plurality of parts of 1 g (dry weight basis) of solid microbial inoculant were taken and respectively inoculated into fresh 50 mL inorganic salt media respectively containing 100 mg/L n-hexane, 100 mg/L pentane, 100 mg/L chlorobenzene and 100 mg/L tetrahydrofuran, and the culture medium was placed into a shaker at a temperature of 30° C. and a revolving speed of 160 r/min for culture, and the degradation rate of the n-hexane, the pentane, the chlorobenzene and the tetrahydrofuran was determined; and a bottle stopper of the inorganic salt medium was opened at the end of each degradation, 100-200 mg/L contaminants were then added into the culture medium, and the degradation performance was monitored, wherein results are shown in FIG. 3, and a pH value in the bottle is shown in Table 2 when no degradation effect was observed in a shake flask.

TABLE 2
Summary table of pH in shake flask without degradation effect

| Contaminant | Chlorobenzene | Tetrahydrofuran | n-hexane | Pentane |
|---|---|---|---|---|
| pH | 4.6 | 5.7 | 5.4 | 5.4 |

It could be seen that the complex microbial inoculant had good continuous contaminant degradation performance. After the first complete degradation of 100 mg/L pollutants by the microbial inoculant, 100 mg/L chlorobenzene may be thoroughly degraded within 6 to 8 hours in subsequent degradation cycles. After repeating this process 10 times, the contaminant concentration was elevated to 200 mg/L, and the contaminants could be thoroughly degraded within 8 to 10 hours. During the 16th consecutive degradation test, the chlorobenzene in the shake flask could not be thoroughly degraded, with the pH in the shake flask determined at 4.6. Subsequently, for 100 mg/L tetrahydrofuran, n-hexane, and pentane, complete degradation could be achieved within 24 to 36 hours, and it took approximately 6 to 8 repetitions before no degradation effect was observed. The pH values in the shake flask were 5.7, 5.4, and 5.4, respectively.

### 4. Contaminant Concentration Tolerance Test of Complex Microbial Inoculant

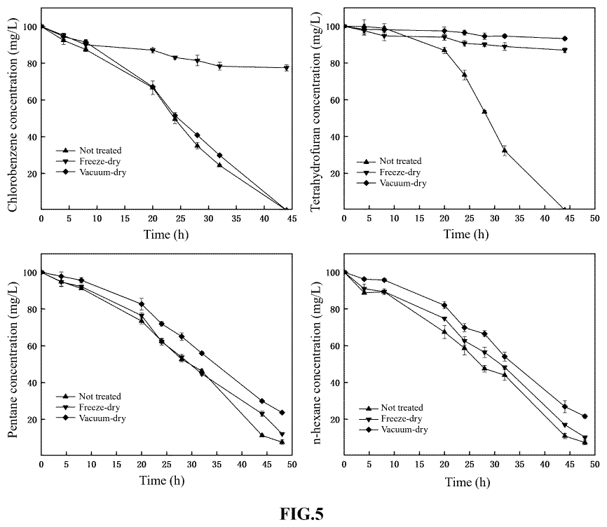

A plurality of parts of 1 g (dry weight basis) of solid microbial inoculant were taken and respectively inoculated into fresh 50 mL inorganic salt media respectively containing 100 mg/L n-hexane, 200 mg/L pentane, 300 mg/L chlorobenzene and 400 mg/L tetrahydrofuran, and the culture medium was placed into a shaker at a temperature of 30° C. and a revolving speed of 160 r/min for culture, and the degradation rate of the n-hexane, the pentane, the chlorobenzene and the tetrahydrofuran was determined; and a bottle stopper of the inorganic salt medium was opened at the end of each degradation, 100-200 mg/L contaminants were then added into the culture medium, and the degradation performance was monitored, wherein results are shown in FIG. 4.

It could be seen that the complex microbial inoculant had good contaminant concentration tolerance. 100 mg/L and 200 mg/L contaminants could be thoroughly degraded within 50 hours, while the contaminants with concentrations of 300 mg/L and 400 mg/L achieved the degradation rate of 90% or above within 65 hours.

### 5. Impact of Different Treatment Methods on Degradation Effect of Solid Complex Microbial Inoculant

The prepared solid complex microbial inoculant is treated in three methods: freeze-drying, vacuum-drying (30° C.) and no dehydration treatment. Specific experimental steps are as follows: the prepared solid complex microbial inoculant was equally divided into three parts: one part was not treated; one part was freeze-dried under vacuum for dehydration for 48 hours; the other part was dried under vacuum at 30° C. for 48 hours, and 1 g (dry weight basis) of each treated sample was inoculated into a fresh 50 mL inorganic salt medium containing 100 mg/L n-hexane, pentane, chlorobenzene, and tetrahydrofuran, and cultured in a shaker at a temperature of 30° C. and a revolving speed of 160 r/min to determine the degradation rate of the n-hexane, the pentane, the chlorobenzene, and the tetrahydrofuran, and results are shown in FIG. 5. The results showed that the untreated group had the best degradation effect on contaminants.

### 6. Impact of Different Deposition Temperatures on Degradation Effect of Complex Microbial Inoculant

On the basis of the performance test 5, the vacuum-dried (30° C.) and untreated solid complex microbial inoculant was further divided into 2 parts, one part was deposited at 4° C. and the other part was deposited at 25° C., and a plurality of parts of 1 g (dry weight basis) of samples were taken out on Day 7, Day 15 and Day 30 respectively for carrying out contaminant degradation experiments, experimental steps were carried out according to the performance test “3. Continuous contaminant degradation test of complex microbial inoculant, and results are shown in Table 3. The results showed that for the n-hexane and the pentane, the best deposition effect was achieved without treatment at 25° C., and for the tetrahydrofuran and the chlorobenzene, there was no difference in the contaminant degradation effect between two treatment methods and deposition conditions.

TABLE 3
Removal effect of various deposition temperatures over time

| | n-hexane | | | | Pentane | | | | Tetrahydrofuran | | | | Chlorobenzene | | | |
|---|---|---|---|---|---|---|---|---|---|---|---|---|---|---|---|---|
| | Vacuum- | | Not | | Vacuum- | | Not | | Vacuum- | | Not | | Vacuum- | | Not | |
| | dry | | treated | | dry | | treated | | dry | | treated | | dry | | treated | |
| Temperature (° C.) | 4 | 25 | 4 | 25 | 4 | 25 | 4 | 25 | 4 | 25 | 4 | 25 | 4 | 25 | 4 | 25 |
| Removal | 90 | 90 | 94 | 94 | 92 | 92 | 95 | 95 | 14 | 14 | 100 | 100 | 100 | 100 | 100 | 100 |
| rate for 7 d | | | | | | | | | | | | | | | | |
| (%) | | | | | | | | | | | | | | | | |
| Removal | 86 | 89 | 93 | 94 | 91 | 91 | 93 | 94 | 10 | 10 | 100 | 100 | 100 | 100 | 100 | 100 |
| rate for 15 d | | | | | | | | | | | | | | | | |
| (%) | | | | | | | | | | | | | | | | |
| Removal | 84 | 87 | 91 | 94 | 90 | 90 | 92 | 93 | 8 | 8 | 100 | 100 | 100 | 100 | 100 | 100 |
| rate for 30 d | | | | | | | | | | | | | | | | |
| (%) | | | | | | | | | | | | | | | | |

1. A method for preparing a complex microbial inoculant for exhaust gas treatment based on aerobic composting, comprising
selecting Pseudomonas mendocina NX-1, Stenotrophomonas sp. HY-2, Rhodococcus sp. YZ-1, Ralstonia sp. XZW-1, and Pseudomonas oleovorans DT4, wherein the Pseudomonas mendocina NX-1 and the Stenotrophomonas sp. HY-2 are screened through n-hexane serving as a carbon source, the Rhodococcus sp. YZ-1 is screened through pentane serving as a carbon source, the Ralstonia sp. XZW-1 is screened through chlorobenzene serving as a carbon source, and the Pseudomonas oleovorans DT4 is screened through tetrahydrofuran serving as a carbon source; inoculating the above strains into an inorganic salt solid medium, then respectively inoculating the five strains into an LB broth for culture to obtain an inoculum solution, inoculating the inoculum solution onto kitchen wastes serving as a culture medium, introducing organic exhaust gases through aerobic composting, and carrying out a domestication and culture process to obtain the complex microbial inoculant; wherein
the Pseudomonas mendocin NX-1 is deposited in the China Center for Type Culture Collection, located at the University of Wuhan, 430072, Wuhan, China, with an accession number of the deposit: CCTCC No. M2015114;
the Stenotrophomonas sp. HY-2 is deposited in the China Center for Type Culture Collection, located at the University of Wuhan, 430072, Wuhan, China, with an accession number of the deposit: CCTCC No. M2018714;
the Rhodococcus sp. YZ-1 is deposited in the China Center for Type Culture Collection, located at the University of Wuhan, 430072, Wuhan, China, with an accession number of the deposit: CCTCC No. M20221106;
the Ralstonia sp. XZW-1 is deposited in the China Center for Type Culture Collection, located at the University of Wuhan, 430072, Wuhan, China, with an accession number of the deposit: CCTCC No. M2022557; and
the Pseudomonas oleovorans DT4 is deposited in the China Center for Type Culture Collection, located at the University of Wuhan, 430072, Wuhan, China, with an accession number of the deposit: CCTCC No. M209151.

2. The method for preparing the complex microbial inoculant for exhaust gas treatment based on aerobic composting according to claim 1, wherein
the inoculum solution is prepared by respectively inoculating the five strains for culture: the Pseudomonas mendocin NX-1, the Stenotrophomonas sp. HY-2, the Rhodococcus sp. YZ-1, the Ralstonia sp. XZW-1 and the Pseudomonas oleovorans DT4, followed by centrifuging, washing, diluting five bacterial suspensions in a logarithm growth period, and mixing five yielded bacterial solutions.

3. The method for preparing the complex microbial inoculant for exhaust gas treatment based on aerobic composting according to claim 2, wherein
the inoculum solution is added to the kitchen wastes in batches, and during the aerobic composting process, an amount of the inoculum solution of the complex microbial inoculant that is inoculated every time is 2% (V/W) of a dry weight of the kitchen wastes.

4. The method for preparing the complex microbial inoculant for exhaust gas treatment based on aerobic composting according to claim 1, wherein
the organic exhaust gases are introduced at the end of a high temperature period of the aerobic composting.

5. The method for preparing the complex microbial inoculant for exhaust gas treatment based on aerobic composting according to claim 1, wherein
during the aerobic composting process, stirring is not performed for the first 7 days, and is then performed every 2 days following 7 days.

6. The method for preparing the complex microbial inoculant for exhaust gas treatment based on aerobic composting according to claim 1, wherein
besides the kitchen wastes, a conditioner and a kitchen fermentation microbial inoculant are further added during the aerobic composting process.

7. A complex microbial inoculant, wherein the complex microbial inoculant is prepared through the method as claimed in claim 1, and comprises Pseudomonas mendocin NX-1, Stenotrophomonas sp. HY-2, Rhodococcus sp. YZ-1, Ralstonia sp. XZW-1, and Pseudomonas oleovorans DT4.

8. An apparatus for preparing the complex microbial inoculant as claimed in claim 7, comprising
a gas distribution system for preparing exhaust gases and a solid fermentation tank connected with the gas distribution system for treating the exhaust gases, wherein
the gas distribution system comprises an air compressor, and an exhaust gas supply bottle and a gas mixing bottle, the exhaust gas supply bottle and the gas mixing bottle are sequentially connected with the air compressor, one side of the air compressor is connected with the exhaust gas supply bottle, the other side of the air compressor is connected with the gas mixing bottle, a thermostat is arranged on an outer wall of the exhaust gas supply bottle, and a temperature probe is arranged in the exhaust gas supply bottle; and
the solid fermentation tank comprises a motor, a stirring apparatus is arranged in the solid fermentation tank and connected with the motor, a temperature control jacket is arranged on an outer wall of the solid fermentation tank, and a gas distribution plate is arranged on an inner wall of the solid fermentation tank.

9. The apparatus according to claim 8, wherein
a microbial filter is arranged between the gas distribution system and the solid fermentation tank, a mass flowmeter is arranged between the air compressor and the exhaust gas supply bottle, and a rotameter is arranged between the gas mixing bottle and the microbial filter.

10. Application of the complex microbial inoculant prepared through the method for preparing the complex microbial inoculant for exhaust gas treatment based on aerobic composting as claimed in claim 1.
